(19) United States
(12) Patent Application Publication
(10) Pub. No.: US 2017/0027949 A1
(43) Pub. Date: Feb. 2, 2017

(54) ASSOCIATION BETWEEN 8-CYCLOPROPYL-3-[2-(3-FLUOROPHENYL)ETHYL]-7,8-DIHYDRO-3H-[1,3]OXAZINO[6,5-g][1,2,3]BENZOTRIAZINE-4,9-DIONE AND AN ACETYLCHOLINESTERASE INHIBITOR, AND PHARMACEUTICAL COMPOSITIONS CONTAINING IT

(71) Applicant: LES LABORATOIRES SERVIER, SURESNES CEDEX (FR)

(72) Inventors: Sylvie BRETIN, ANTONY (FR); Maria PUEYO, PARIS (FR)

(21) Appl. No.: 15/302,245

(22) PCT Filed: Apr. 3, 2015
(86) PCT No.: PCT/FR2015/050879

§ 371 Date: Oct. 6, 2016

(30) Foreign Application Priority Data
Apr. 7, 2014 (FR) 1453046

(51) Int. Cl. A61K 31/5365; A61K 9/20; A61K 31/27; A61K 31/55; A61K 31/445

(52) U.S. Cl. CPC A61K 31/5365; A61K 31/55; A61K 31/445; A61K 31/27; A61K 9/2059; A61K 9/2013; A61K 9/2018; A61K 9/2054

(57) ABSTRACT

Association between 8-cyclopropyl-3-[2-(3-fluorophenyl)ethyl]-7,8-dihydro-3H-[1,3]-oxazino[6,5-g][1,2,3]benzotriazine-4,9-dione of formula (I):

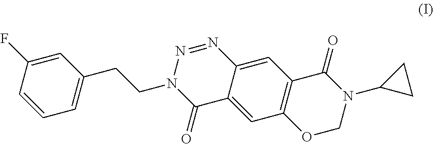 [SMILES: O=C1c2cc3nnn(CCc4cccc(F)c4)c(=O)c3cc2OCN1C1CC1]

or an addition salt thereof with a pharmaceutically acceptable acid or base, and an acetylcholinesterase inhibitor.

Medicinal products containing the same which are useful in treating cognitive disorders associated with cerebral ageing and with neurodegenerative diseases.

The present invention relates to a new association between 8-cyclopropyl-3-[2-(3-fluorophenyl)ethyl]-7,8-dihydro-3H-[1,3]oxazino[6,5-g][1,2,3]benzotriazine-4,9-dione of formula (I):

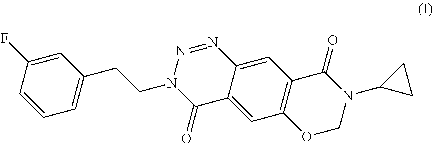 [SMILES: O=C1c2cc3nnn(CCc4cccc(F)c4)c(=O)c3cc2OCN1C1CC1]

or an addition salt thereof with a pharmaceutically acceptable acid or base, and an acetylcholinesterase inhibitor for obtaining pharmaceutical compositions for use in the treatment of cognitive disorders associated with cerebral ageing and with neurodegenerative diseases.

8-Cyclopropyl-3-[2-(3-fluorophenyl)ethyl]-7,8-dihydro-3H-[1,3]oxazino[6,5-g][1,2,3]-benzotriazine-4,9-dione is a glutamate AMPA receptor positive allosteric modulator described in patent application WO 2008/085506. More precisely, the compound of formula (I) possesses procognitive properties, improves synaptic plasticity and exhibits neuroprotective properties, giving it an activity which is of interest in the treatment of disorders of the central nervous system and, more especially, in the treatment of cognitive deficits associated with cerebral ageing and with neurodegenerative diseases.

The present invention relates to the association between 8-cyclopropyl-3-[2-(3-fluorophenyl)ethyl]-7,8-dihydro-3H-[1,3]oxazino[6,5-g][1,2,3]benzotriazine-4,9-dione of formula (I), or addition salts thereof with a pharmaceutically acceptable acid or base, and an acetylcholinesterase inhibitor, as well as the properties thereof which are of interest for the treatment of cognitive disorders associated with cerebral ageing and with neurodegenerative diseases.

Neurodegenerative diseases associated with cerebral ageing, such as Alzheimer's disease, are characterised by memory disorders and cognitive dysfunctions. Cognitive disorders are generally associated with a reduction in the ability of neurons to synthesise and release certain neurotransmitters such as, for example, glutamate and acetylcholine (Robbins et al., Trends in Pharmacol. Sci. 2006(3), 27, 141-148). In addition, a progressive loss of synaptic plasticity and of neuronal processes is observed, that neuronal loss being accelerated in certain specific regions of the brain. Pathophysiological studies have clearly shown that a deficit of glutamatergic neurotransmission is closely associated with the development of Alzheimer's disease (Advokat et al., Neuroscience & Biobehav. Rev. 1992, 16, 13-24; Francis et al., Progress in Neurobiology 1992, 39(5), 517-545).

In the glutamatergic system, the AMPA (“α-amino-3-hydroxy-5-methyl-4-isoxazole-propionic acid”) receptor appears to be the receptor that is most involved in the phenomena of physiological neuronal excitability and, especially, in phenomena involved in the process of memorisation. For example, learning has been shown to be associated with the increased binding of AMPA to its receptor in the hippocampus, one of the regions of the brain that are essential to cognitive processes.

In parallel, a progressive degeneration of the cholinergic neurons is observed in the course of Alzheimer's disease. Acetylcholinesterase inhibitors such as donepezil are currently being used in the symptomatic treatment of Alzheimer's disease in order to limit the reduction in acetylcholine levels in the brain by blocking the action of acetylcholinesterase. It has been shown that acetylcholinesterase inhibitors, like AMPA receptor positive allosteric modulators, enable the cognitive properties to be improved in various animal models of episodic memory and working memory (Black, Psychopharmacol. 2005, 179, 154-163; O'Neill et al., IDrugs 2007, 10(3), 185-192; Yuede et al., Behav. Pharmacol., 2007, 18(5-6), 347-363). Improvement in cognitive functions may therefore be based on two types of strategy, targeting either the AMPA receptors or glutamate, or acetylcholine.

Surprisingly, the present invention has shown that the effects of acetylcholinesterase inhibitors are potentiated by those of 8-cyclopropyl-3-[2-(3-fluorophenyl)ethyl]-7,8-dihydro-3H-[1,3]oxazino[6,5-g][1,2,3]benzotriazine-4,9-dione or of addition salts thereof with a pharmaceutically acceptable acid or base. Accordingly, the coadministration of those compounds may allow the cognitive performances of patients to be improved as compared with the simple administration of an acetylcholinesterase inhibitor, but without observing or increasing the harmful effects associated with the treatment (in particular, gastrointestinal disorders such as nausea or diarrhoea, headaches or fatigue). In other words, treatments using therapeutic doses of acetylcholinesterase inhibitor that are lower than those conventionally employed in mono-administration therefore become envisageable, with equivalent or even superior cognitive performances and fewer harmful effects.

More surprisingly, the association between 8-cyclopropyl-3-[2-(3-fluorophenyl)ethyl]-7,8-dihydro-3H-[1,3]oxazino[6,5-g][1,2,3]benzotriazine-4,9-dione, or an addition salt thereof, administered in a subactive dose, and an acetylcholinesterase inhibitor which is also administered in a subactive dose, causes a synergistic effect with good performances on cognition.

These effects, which were not foreseeable, make it possible to envisage using associations between 8-cyclopropyl-3-[2-(3-fluorophenyl)ethyl]-7,8-dihydro-3H-[1,3]oxazino[6,5-g]-[1,2,3]benzotriazine-4,9-dione, or an addition salt thereof, and an acetylcholinesterase inhibitor in the treatment of cognitive disorders associated with cerebral ageing and with neurodegenerative diseases. Cognitive disorders associated with Alzheimer's disease are especially targeted. More especially, cognitive disorders associated with Alzheimer's disease in patients having symptoms of depression are targeted.

Preferably, 8-cyclopropyl-3-[2-(3-fluorophenyl)ethyl]-7,8-dihydro -3H-[1,3]oxazino-[6,5-g][1,2,3]benzotriazine-4,9-dione is used in the form of a base within the context of the invention.

Among the acetylcholinesterase inhibitors according to the invention there will be chosen from among physostigmine, rivastigmine, galantamine, donepezil, tacrine and huperzine A. According to a preferred embodiment, the acetylcholinesterase inhibitor is chosen from donepezil, rivastigmine and galantamine. Donepezil is preferably used in the form of a hydrochloride, rivastigmine in the form of a hydrogen tartrate and galantamine in the form of a hydrobromide. Advantageously, the acetylcholinesterase inhibitor is donepezil.

More especially, the association of 8-cyclopropyl-3-[2-(3-fluorophenyl)ethyl]-7,8-dihydro-3H-[1,3]oxazino[6,5-g][1,2,3]benzotriazine-4,9-dione and an acetylcholinesterase inhibitor chosen from donepezil, rivastigmine and galantamine is used in the treatment of cognitive disorders associated with Alzheimer's disease and, more especially, cognitive disorders associated with Alzheimer's disease in patients having symptoms of depression.

The invention relates also to the use of the association between 8-cyclopropyl-3-[2-(3-fluorophenyl)ethyl]-7,8-dihydro-3H-[1,3]oxazino[6,5-g][1,2,3]benzotriazine-4,9-dione, or addition salts thereof with a pharmaceutically acceptable acid or base, and an acetylcholinesterase inhibitor for obtaining pharmaceutical compositions for the treatment of cognitive disorders associated with cerebral ageing and with neurodegenerative diseases.

More especially, the invention relates to the use of the association between 8-cyclopropyl-3-[2-(3-fluorophenyl)ethyl]-7,8-dihydro-3H-[1,3]oxazino[6,5-g][1,2,3]benzotriazine-4,9-dione, or addition salts thereof with a pharmaceutically acceptable acid or base, and an acetylcholinesterase inhibitor chosen from donepezil, rivastigmine and galantamine, for obtaining pharmaceutical compositions for the treatment of cognitive disorders associated with Alzheimer's disease and, more especially, cognitive disorders associated with Alzheimer's disease in patients having symptoms of depression.

The invention relates also to pharmaceutical compositions comprising the association between 8-cyclopropyl-3-[2-(3-fluorophenyl)ethyl]-7,8-dihydro-3H-[1,3]oxazino[6,5-g]-[1,2,3]benzotriazine-4,9-dione, or addition salts thereof with a pharmaceutically acceptable acid or base, and an acetylcholinesterase inhibitor in combination with one or more pharmaceutically acceptable excipients.

Advantageously, the invention relates to pharmaceutical compositions comprising the association between 8-cyclopropyl-3-[2-(3-fluorophenyl)ethyl]-7,8-dihydro-3H-[1,3]-oxazino[6,5-g][1,2,3]benzotriazine-4,9-dione, or addition salts thereof with a pharmaceutically acceptable acid or base, and an acetylcholinesterase inhibitor chosen from donepezil, rivastigmine and galantamine, in combination with one or more pharmaceutically acceptable excipients.

In the pharmaceutical compositions according to the invention, the fraction by mass of active ingredients (mass of the active ingredients over the total mass of the composition) is from 5 to 50%.

Among the pharmaceutical compositions according to the invention there may be mentioned more especially those that are suitable for administration by the oral, parenteral and especially intravenous, per- or trans-cutaneous, nasal, rectal, perlingual, ocular or respiratory route and more specifically tablets, dragées, sublingual tablets, gelatin capsules, glossettes, capsules, lozenges, injectable preparations, aerosols, eye or nasal drops, suppositories, creams, ointments, dermal gels, transdermal patches, etc.

In addition to the 8-cyclopropyl-3-[2-(3-fluorophenyl)ethyl]-7,8-dihydro-3H-[1,3]oxazino[6,5-g][1,2,3]benzotriazine-4,9-dione and the acetylcholinesterase inhibitor compound, the pharmaceutical compositions according to the invention comprise one or more excipients or carriers chosen from diluents, lubricants, binders, disintegrators, stabilisers, preservatives, absorbents, colourings, sweeteners, flavourings, etc.

Examples which may be mentioned, without implying any limitation, include:
for the diluents: lactose, dextrose, sucrose, mannitol, sorbitol, cellulose, glycerin;
for the lubricants: silica, talc, stearic acid and its magnesium and calcium salts, polyethylene glycol;
for the binders: aluminium and magnesium silicate, starch, gelatin, tragacanth, methylcellulose, sodium carboxymethylcellulose and polyvinylpyrrolidone;
for the disintegrators: agar, alginic acid and its sodium salt, effervescent mixtures.

The compounds of the association can be administered simultaneously or in succession. The preferred administration route is the oral route. Another advantageous administration route is the transdermal patch. The corresponding pharmaceutical compositions can permit the immediate or delayed release of the active ingredients. Moreover, the compounds of the association can be administered in the form of two separate pharmaceutical compositions, each comprising one of the active ingredients, or alternatively in the form of a single pharmaceutical composition in which the active ingredients are mixed.

The preferred pharmaceutical compositions are tablets.

The dosage used varies according to the sex, age and weight of the patient, the administration route, the nature of the disorder and of any associated treatments and ranges from 1 to 200 mg of equivalents of free base of 8-cyclopropyl-3-[2-(3-fluorophenyl)ethyl]-7,8-dihydro-3H-[1,3]oxazino[6,5-g][1,2,3]benzotriazine-4,9-dione per 24 hours, and more preferably from 5 to 50 mg per day. The dose of the acetylcholinesterase inhibitor will be equal to or less than that used when it is administered on its own. In the case of donepezil, the dosage is from 0.5 to 30 mg per day, the preferred daily doses being from 5 to 10 mg. In the case of rivastigmine, the dosage is from 0.5 to 20 mg per day, the preferred daily doses being from 1.5 to 14 mg. In the case of galantamine, the dosage is from 1 to 30 mg per day, the preferred daily doses being from 8 to 24 mg.

### EXAMPLE A

### Experiment in a Model of Episodic Memory, the Contextual Serial Discrimination Test

The effects of 8-cyclopropyl-3-[2-(3-fluorophenyl)ethyl]-7,8-dihydro-3H-[1,3]oxazino-[6,5-g][1,2,3]benzotriazine-4,9-dione and of donepezil (the latter used in the form of a hydrochloride), administered on their own or in combination, were studied using a contextual serial discrimination test in C57BL/6 mice having an average age of 14-15 months (n=12 per group) (Célérier et al., Learn Mem. 2004, 11(2), 196-204; Tronche et al., Behav. Brain Res. 2010, 215(2), 255-260). In this model, the middle-aged mice exhibit a specific dysfunction of contextual memory as compared with the young mice, without any defect of spatial memory. This model is relevant for evaluating the effects of products in Alzheimer's disease because patients affected by this dementia also exhibit, at a very early stage, contextual episodic memory disorders (Gold et al., Expert Rev. Neurother. 2008, 8(12), 1879-1891).

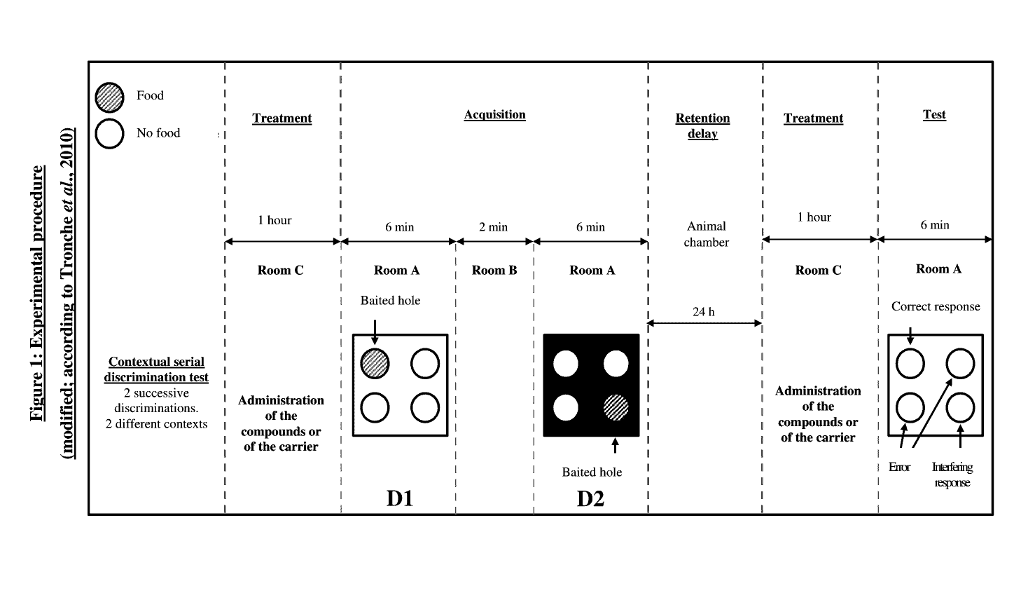

The mice, placed in a high-sided box, learn two types of consecutive spatial discriminations (D1: white floor then D2: black floor) on a floor having four holes, in which only one of the holes is baited, opposite holes being baited in D1 and D2. Each discrimination is performed on a specific floor (white or black), which constitutes the internal context specific to each discrimination. Twenty-four hours after the learning step, the mice are returned to the white contextual floor and the following parameters are measured:
the percentage of correct responses (i.e. % of head dips in the hole that had been baited during the acquisition exercise on the white floor),
the percentage of interfering responses (i.e. % of head dips in the hole that had been baited during the acquisition exercise on the black floor, the last context presented to the mice),
and the percentage of errors (i.e. % of head dips in the two holes that had not been baited during the acquisition, either on the white floor or on the black floor) (see FIG. 1).

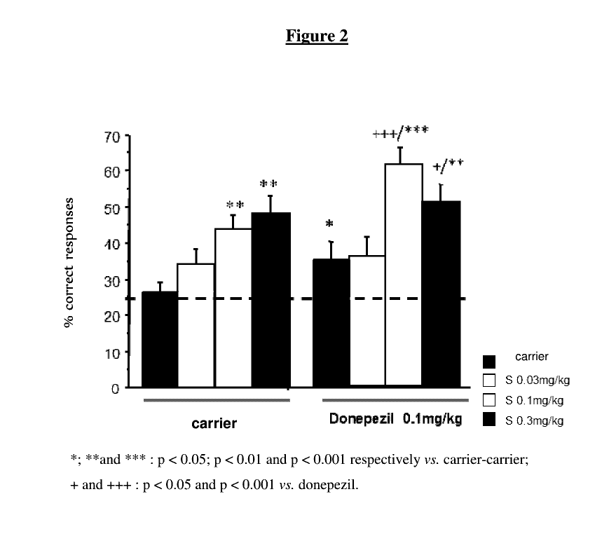

The results show that the middle-aged mice treated with the carrier exhibit a percentage of correct responses which is close to the level of chance in this test on 4-hole plates (≈25%). After chronic treatment for 9 days with donepezil hydrochloride (0.1 mg/kg of base per os), a slight non-significant increase in the percentage of correct responses is observed relative to the carrier (35.5% versus 26.4%, see FIG. 2). By contrast, the level of correct responses increases significantly by more than 40% relative to the carrier after chronic treatment for 9 days with 8-cyclopropyl-3-[2-(3-fluorophenyl)ethyl]-7,8-dihydro-3H-[1,3]oxazino[6,5-g][1,2,3]benzotriazine-4,9-dione at doses of 0.1 and 0.3 mg/kg per os (43.7% correct responses at 0.1 mg/kg and 48% correct responses at 0.3 mg/kg) (compound denoted S in FIG. 2). Finally, administration of the association of 8-cyclopropyl-3-[2-(3-fluorophenyl)ethyl]-7,8-dihydro-3H-[1,3]oxazino[6,5-g][1,2,3]-benzotriazine-4,9-dione (0.1 mg/kg of base per os) with donepezil (0.1 mg/kg of base per os) leads to a significant increase in the level of correct responses since those responses are than 100% greater relative to the carrier on its own (61.1% correct responses with this association versus 26.4% with the carrier on its own). These results show a clear potentiation of the effects of donepezil in the presence of 8-cyclopropyl-3-[2-(3-fluorophenyl)ethyl]-7,8-dihydro-3H-[1,3]oxazino[6,5-g][1,2,3]benzotriazine-4,9-dione.

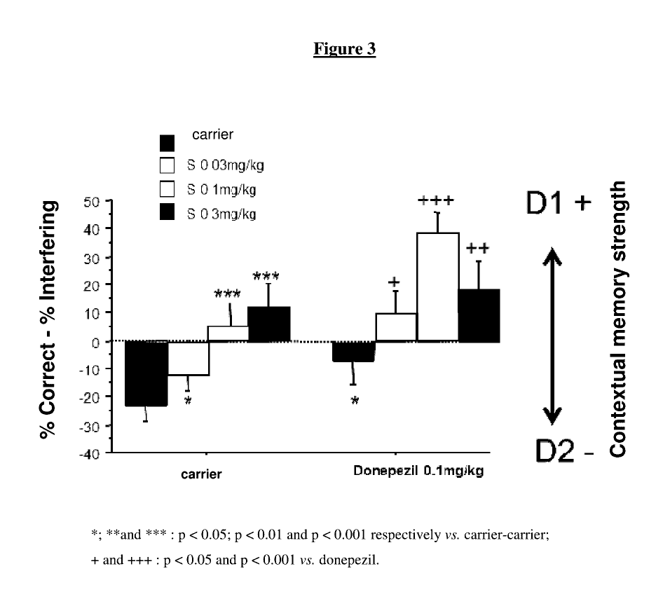

In addition, a very good correlation between the increase in the level of correct responses and the reduction in the level of interfering responses is observed, thus confirming the specific effect of each compound and of their association on contextual memory. Accordingly, administration of the association of 8-cyclopropyl-3-[2-(3-fluorophenyl)ethyl]-7,8-dihydro-3H-[1,3]oxazino[6,5-g][1,2,3]benzotriazine-4,9-dione (0.1 mg/kg of base per os) with donepezil (0.1 mg/kg of base per os) significantly increases the contextual memory strength (correct responses—interfering responses) as compared with that observed with the compounds on their own. This increase observed for the association cannot be explained by a mere additivity of the effects of the compounds administered on their own and shows a synergy of activity of the two compounds when they are coadministered (see FIG. 3).

The results clearly show that the administration of 8-cyclopropyl-3-[2-(3-fluorophenyl)ethyl]-7,8-dihydro-3H-[1,3]oxazino[6,5-g][1,2,3]benzotriazine-4,9-dione and of donepezil in association makes it possible to obtain a considerable synergistic effect which is wholly unexpected. Moreover, the pharmacokinetic analyses have shown that there was no pharmacokinetic-type interaction between the two treatments which might justify or interfere with the synergistic effect described above.

### EXAMPLE B

### Irwin's Primary Observation Test

The effects in terms of safety of 8-cyclopropyl-3-[2-(3-fluorophenyl)ethyl]-7,8-dihydro-3H-[1,3]oxazino[6,5-g][1,2,3]benzotriazine-4,9-dione and of donepezil (the latter in the form of a hydrochloride), administered on their own or in association, were studied using Irwin's primary observation test in C57BL/6 mice (n=4 individuals per group). Behavioural changes, physiological and neurotoxic symptoms, rectal temperature and also pupil diameter were recorded using a standardised observation grid derived from that of Irwin.

It was observed that 8-cyclopropyl-3-[2-(3-fluorophenyl)ethyl]-7,8-dihydro-3H-[1,3]-oxazino[6,5-g][1,2,3]benzotriazine-4,9-dione (from 3 to 100 mg/kg of base per os) and donepezil (at 0.3 and 1 mg/kg of base per os), administered acutely on their own or in coadministration, do not induce any change in the mice according to the standardised observation grid. At a higher dose (3 and 10 mg/kg of base per os), donepezil administered on its own induces shaking, slight to moderate sedation, a loss of muscle tone and a decrease in reactivity to touch. No potentiation of the effects observed with donepezil at 3 and 10 mg/kg was observed when it is coadministered with 8-cyclopropyl-3-[2-(3-fluorophenyl)ethyl]-7,8-dihydro-3H-[1,3]oxazino[6,5-g][1,2,3]benzotriazine-4,9-dione (from 3 to 100 mg/kg of base per os). In addition, in this study, the pharmacokinetic analyses showed that there was no pharmacokinetic-type interaction between the two treatments which might justify or interfere with the observations described above.

In conclusion, the results presented above show a synergy of activity between 8-cyclopropyl-3-[2-(3-fluorophenyl)ethyl]-7,8-dihydro-3H-[1,3]oxazino[6,5-g][1,2,3]-benzotriazine-4,9-dione and donepezil in terms of cognitive performances, with a good safety profile and without pharmacokinetic interaction.

### EXAMPLE C

### Study of the Excitatory Post-Synaptic Potentials (EPSP) Induced by Electrical Stimulation in Alert Mice

8-Cyclopropyl-3-[2-(3-fluorophenyl)ethyl]-7,8-dihydro-3H-[1,3]oxazino[6,5-g][1,2,3]-benzotriazine-4,9-dione and rivastigmine were evaluated electrophysiologically in alert animals after a single oral administration and in coadministration at subactive doses of the two compounds in order to study a synergistic potential between those two compounds in the hippocampus. Studying EPSPs induced by electrical stimulation in alert mice makes it possible to study physiologically an electrophysiological phenomenon which is the basis of neuronal activity and connected to the processes of memorisation and learning. Learning and memory in fact require changes in the neuronal circuits. Therefore, the facilitation of synaptic responses by means of a product or an association of products by acting on the excitatory post-synaptic potentials would make it possible to facilitate learning or to counteract age-related memory deficits or memory deficits occurring in the case of neurodegenerative diseases such as Alzheimer's disease (Shapiro, Arch. Neurol. 2001, 58, 874-881).

The studies were carried out as follows:

3-month-old C57BL/6 mice are anaesthetised with 0.8-3% halothane delivered by means of a mask. Once anaesthetised, stimulation and recording electrodes are inserted, respectively, in the perforant pathway and the dentate gyrus of the hippocampus of the animals. Following implantation of the electrodes, the mice are kept in cages for from 5 to 7 days with free access to food and water in order to allow complete recovery. Each implanted animal is alone in a cage until the end of the experiment.

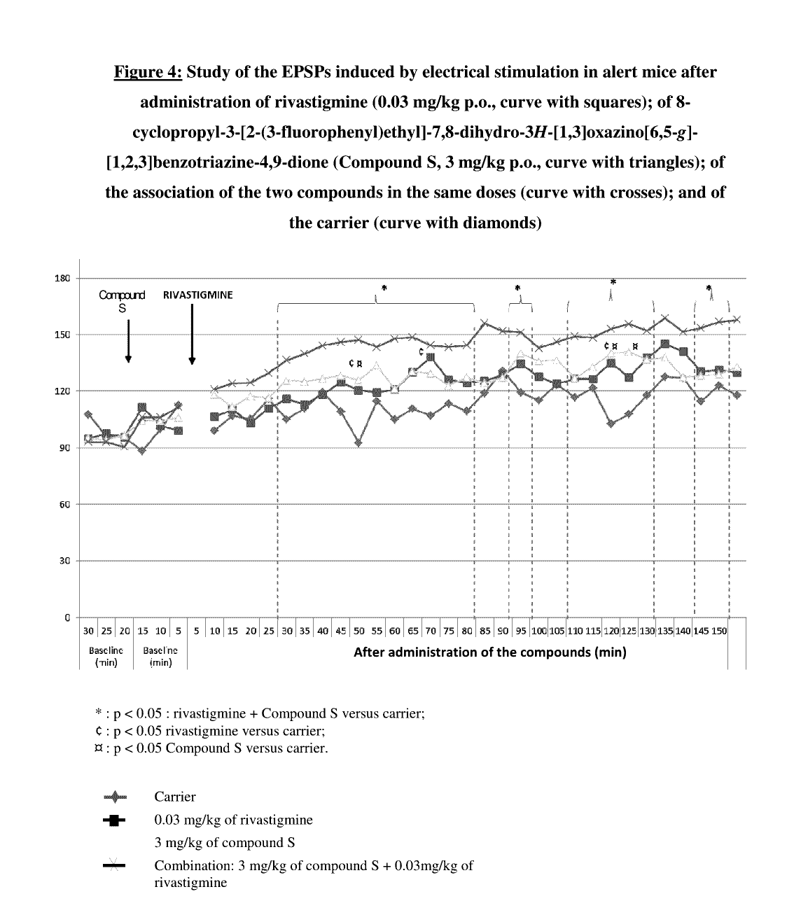

After recovery, a stable baseline of evoked responses is induced using paired pulses (pulse duration 100 μs, negative-positive) administered at 3 pulses/minute to the stimulation electrode situated in the perforant pathway. The intervals between pulses are fixed at 40 ms (Gruart et al., J. Neurosci. 2006, 24, 1077-1087). The field EPSPs are monitored until a stable baseline is obtained (for 30 minutes). The products or the carrier are then administered, and the evoked field potentials are recorded. The compounds are administered per os on their own or in coadministration in the following doses: rivastigmine (0.03 mg/kg, p.o.); 8-cyclopropyl-3-[2-(3-fluorophenyl)ethyl]-7,8-dihydro-3H-[1,3]oxazino[6,5-g][1,2,3]benzotriazine-4,9-dione (compound denoted S in FIG. 4; 3 mg/kg, p.o.); carrier (1% (w/v) hydroxyethylcellulose and 1% (v/v) polysorbate 80 in distilled water). For reasons of kinetics associated with the tₘₐₓ of each of the products, administration of the compounds was carried out as follows: 8-cyclopropyl-3-[2-(3-fluorophenyl)ethyl]-7,8-dihydro-3H-[1,3]oxazino[6,5-g][1,2,3]benzotriazine-4,9-dione and rivastigmine were administered by gavage, 45 minutes and 30 minutes, respectively, before induction of the paired pulses as described above. In order that the groups of animals are homogeneous, the animals receiving only one of the two products receive carrier during the second administration. The evoked potentials are then recorded for approximately 2.5 hours after the last administration of compound or carrier.

The results show that, at the baseline level, all the groups are identical (non-significant difference) (see FIG. 4, “baseline”). Following administration of the various compounds, the results are statistically different (two-way ANOVA between the treatments and the sessions; p <0.001). Post hoc analyses show that:
when administered on its own in a single dose of 3 mg/kg p.o., 8-cyclopropyl-3-[2-(3-fluorophenyl)ethyl]-7,8-dihydro-3H-[1,3]oxazino[6,5-g][1,2,3]benzotriazine-4,9-dione does not induce a significant modification of the amplitudes of the field EPSPs relative to the carrier group, except at 3 isolated times (at 45, 115 and 120 minutes after induction of the paired pulses), indicating that the dose is subactive (see FIG. 4, curve with triangles and Table 1);
when administered on its own in a single dose of 0.03 mg/kg, rivastigmine does not induce a significant modification of the amplitudes of the field EPSPs relative to the carrier group, except at three isolated times (at 45, 65 and 115 minutes after induction of the paired pulses), indicating that the dose is subactive (see FIG. 4, curve with squares and Table 1);
the association of the two compounds, 8-cyclopropyl-3-[2-(3-fluorophenyl)ethyl]-7,8-dihydro-3H-[1,3]oxazino[6,5-g][1,2,3]benzotriazine-4,9-dione in a single dose of 3 mg/kg p.o. and rivastigmine in a single dose of 0.03 mg/kg p.o., induces a significant increase in the amplitudes of the field EPSPs relative to the carrier group over almost all the recording period (especially from 25 to 80 minutes and then from 90 to 95 minutes, then from 105 to 130 minutes and from 140 to 150 minutes after administration of rivastigmine; p<0.05) (see FIG. 4, curve with crosses and Table 1).

TABLE 1
Results of the post hoc analyses

| | | CARRIER | | |
|---|---|---|---|---|
| | | | | 0.03 mg/kg of |
| | | | | rivastigmine + |
| | | 0.03 mg/kg of | 3.0 mg/kg of | 3.0 mg/kg of |
| Time | | rivastigmine | Compound S | Compound S |
| Baseline-30 | | 0.681 | 0.621 | 0.625 |
| Baseline-25 | | 0.987 | 0.997 | 0.997 |
| Baseline-20 | | 0.985 | 1.000 | 0.977 |
| Baseline-15 | | 0.135 | 0.399 | 0.348 |
| Baseline-10 | | 0.869 | 0.989 | 0.992 |
| Baseline-5 | | 0.705 | 0.886 | 0.921 |
| 5 | minutes | 0.725 | 0.277 | 0.183 |
| 10 | minutes | 0.939 | 0.954 | 0.449 |
| 15 | minutes | 0.833 | 0.549 | 0.265 |
| 20 | minutes | 0.861 | 0.960 | 0.633 |
| 25 | minutes | 0.639 | 0.205 | 0.015 |
| 30 | minutes | 0.853 | 0.424 | 0.032 |
| 35 | minutes | 0.894 | 0.738 | 0.081 |
| 40 | minutes | 0.241 | 0.229 | 0.003 |
| 45 | minutes | 0.028 | 0.007 | <0.001 |
| 50 | minutes | 0.660 | 0.244 | 0.034 |
| 55 | minutes | 0.230 | 0.310 | <0.001 |
| 60 | minutes | 0.225 | 0.243 | 0.002 |
| 65 | minutes | 0.014 | 0.118 | 0.002 |
| 70 | minutes | 0.530 | 0.584 | 0.024 |
| 75 | minutes | 0.254 | 0.284 | 0.005 |
| 80 | minutes | 0.904 | 0.856 | 0.003 |
| 85 | minutes | 0.972 | 0.980 | 0.156 |
| 90 | minutes | 0.356 | 0.596 | 0.014 |
| 95 | minutes | 0.526 | 0.197 | 0.043 |
| 100 | minutes | 0.971 | 0.592 | 0.161 |
| 105 | minutes | 0.558 | 0.688 | 0.011 |
| 110 | minutes | 0.632 | 0.601 | 0.056 |
| 115 | minutes | 0.007 | 0.002 | <0.001 |
| 120 | minutes | 0.163 | 0.007 | <0.001 |
| 125 | minutes | 0.251 | 0.243 | 0.006 |
| 130 | minutes | 0.311 | 0.521 | 0.016 |
| 135 | minutes | 0.536 | 0.979 | 0.103 |
| 140 | minutes | 0.323 | 0.337 | 0.001 |
| 145 | minutes | 0.810 | 0.810 | 0.007 |
| 150 | minutes | 0.413 | 0.362 | <0.001 |

The results clearly show that the administration of 8-cyclopropyl-3-[2-(3-fluorophenyl)ethyl]-7,8-dihydro-3H-[1,3]oxazino[6,5-g][1,2,3]benzotriazine-4,9-dione and of rivastigmine in association makes it possible to obtain a considerable and wholly unexpected synergistic effect.

### EXAMPLE D

### Pharmaceutical Composition

Preparation formula for 1000 tablets containing a dose of 5 mg of 8-cyclopropyl-3-[2-(3-fluorophenyl)ethyl]-7,8-dihydro-3H-[1,3]oxazino[6,5-g][1,2,3]benzotriazine-4,9-dione and 10 mg of an acetylcholinesterase inhibitor chosen from donepezil, rivastigmine and galantamine:

| | | |
|---|---|---|
| 8-Cyclopropyl-3-[2-(3-fluorophenyl)ethyl]-7,8-dihydro-3H- | 5 | g |
| [1,3]oxazino[6,5-g][1,2,3]-benzotriazine-4,9-dione | | |
| Acetylcholinesterase inhibitor | 10 | g |
| Maize starch | 20 | g |
| Maltodextrin | 7.5 | g |
| Colloidal silica | 0.2 | g |
| Sodium starch glycolate | 3 | g |
| Magnesium stearate | 1 | g |
| Lactose | 55 | g |

1-15. (canceled)

16. A combination between 8-cyclopropyl-3-[2-(3-fluorophenyl)ethyl]-7,8-dihydro-3H-[1,3]oxazino[6,5-g][1,2,3]benzotriazine-4,9-dione of formula (I): 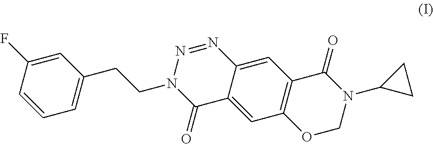 [SMILES: O=C1c2cc3nnn(CCc4cccc(F)c4)c(=O)c3cc2OCN1C1CC1]
or an addition salt thereof with a pharmaceutically acceptable acid or base, and an acetylcholinesterase inhibitor.

17. The combination according to claim 16, wherein the 8-cyclopropyl-3-[2-(3-fluorophenyl)ethyl]-7,8-dihydro-3H-[1,3]oxazino[6,5-g][1,2,3]benzotriazine-4,9-dione is in the form of a base.

18. The combination according to claim 16, wherein the acetylcholinesterase inhibitor is donepezil, rivastigmine or galantamine.

19. The combination according to claim 18, wherein donepezil is in the form of a hydrochloride.

20. The combination according to claim 16, wherein the 8-cyclopropyl-3-[2-(3-fluorophenyl)ethyl]-7,8-dihydro-3H-[1,3]oxazino[6,5-g][1,2,3]-benzotriazine-4,9-dione is present at a dose of from 1 to 200 mg of equivalents of free base.

21. A pharmaceutical composition comprising as active ingredient the combination between 8-cyclopropyl-3-[2-(3-fluorophenyl)ethyl]-7,8-dihydro-3H-[1,3]oxazino[6,5-g][1,2,3]benzotriazine-4,9-dione, or an addition salt thereof with a pharmaceutically acceptable acid or base, and an acetylcholinesterase inhibitor, according to claim 16, in combination with one or more pharmaceutically acceptable excipients.

22. The pharmaceutical composition according to claim 21, wherein 8-cyclopropyl-3-[2-(3-fluorophenyl)ethyl]-7,8-dihydro-3H-[1,3]oxazino[6,5-g][1,2,3]benzotriazine-4,9-dione is present at a dose of from 1 to 200 mg of equivalents of free base.

23. A method of treating a condition selected from cognitive disorders associated with cerebral ageing and with neurodegenerative diseases in a subject in need thereof, comprising administration of an effective amount of the combination according to claim 16, alone or in combination with one or more pharmaceutically acceptable excipients.

24. The method according to claim 23, wherein the condition is cognitive disorders associated with Alzheimer's disease.

25. The method according to claim 24, wherein the subject has symptoms of depression.

26. The method according to claim 23, wherein the 8-cyclopropyl-3-[2-(3-fluorophenyl)ethyl]-7,8-dihydro-3H-[1,3]oxazino[6,5-g][1,2,3]benzotriazine-4,9-dione is administered at a daily dose of from 1 to 200 mg of equivalents of free base.
